(19) United States
(12) Patent Application Publication
(10) Pub. No.: US 2007/0172514 A1
(43) Pub. Date: Jul. 26, 2007

(54) Materials and methods for improving livestock productivity

(76) Inventors: Francis Chi, Kowloon (HK); Gary Kwan Po Wong, Kowloon (HK)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/335,938

(22) Filed: Jan. 20, 2006

(51) Int. Cl. A61K 47/00; A23K 1/165; A61K 31/13

(52) U.S. Cl. 424/442; 426/635; 514/665

(57) ABSTRACT

The subject invention provides methods for improving livestock health. In specific embodiments, the invention provides methods for accelerating and/or augmenting livestock growth; improving immunity; and enhancing fertility in livestock. To do so, the present invention provides materials and methods for administering a cysteamine compound to livestock.

### BACKGROUND OF THE INVENTION

The world's population is increasing rapidly, with estimates of 6 billion to 10 billion people by the year 2050. This increase in population will necessitate a concurrent increase in demand for agricultural meat products. Concurrently, the area of useful agricultural land is shrinking and, in many cases, deteriorating in quality. See Roberts, M., “U.S. Animal Agriculture: Making the Case for Productivity,” AgBioForum, 3(2&3):120-126 (2000). Thus, it is expected that the global demand for animal protein will shortly outpace current animal productive capacity.

To address these issues, a variety of methods have been developed in attempts to bridge the gap between the supply and demand for meat products. For example, much of the world's agricultural livestock is now produced in intensive farming operations. In addition to farming operations, improved livestock nutrition and veterinary care as well as the development of genetic biotechnologies and growth hormone treatments have contributed toward meeting increased consumer demand for animal protein.

It has long been established that growth hormones play an important role in regulating growth of animals. For instance, administering growth hormones in meat producing animals will increase their body weight including their muscle mass. However, there are a number of disadvantages in using growth hormones directly in increasing meat production in these animals.

Firstly, growth hormones from different animals are seldom homogenous and different animals (e.g. mammalian animals) only react to certain types of specific growth hormones. Since suitable exogenous growth hormones are normally extracted from pituitary glands, it is rather difficult and uneconomical to prepare sufficient quantity of suitable exogenous growth hormones for use on a large-scale application. Although exogenous growth hormones can now be prepared using DNA recombinant technology, exogenous growth hormones manufactured by such method are still rather expensive.

Secondly, the administration of exogenous growth hormones into farm animals is normally performed by direct injection, which is inevitably rather costly and difficult to administer in a large farm. Thirdly, it is rather difficult to control the dose administered to produce precisely the desired effect, and an overdose of exogenous growth hormones is likely to be harmful to the animals. Fourthly, residuals of these exogenous growth hormones may be passed to the meat products and subsequently to humans through consumption thereof. Further studies in this regard are required although some scientists are concerned about the negative side effects of these exogenous growth hormones to humans.

Cysteamine is a component of co-enzyme A and works as a physiological regulator. Cysteamine has been used as an additive in feed in promoting growth of meat producing animals. U.S. Pat. No. 4,711,897 discloses animal feed methods and feed compositions comprising cysteamine. However, it has been identified that cysteamine is a fairly sensitive and unstable compound under normal room temperature conditions. For example, cysteamine is readily oxidized when exposed to air or at an elevated temperature. Cysteamine is highly hydroscopic. Also, cysteamine is unpalatable when taken directly by mouth. Further, ingesting cysteamine directly will cause undesirable gastro side effects. For these reasons, the use of cysteamine had for a long time been limited to direct injection of cysteamine-containing solution into meat producing animals. Effective and large scale application of using cysteamine in promoting growth of farm animals has thus been impractical.

Moreover, whether cysteamine can promote productive efficiency (such as increased milk yield per feed unit or increased efficiency in oocyte maturation) and improve carcass composition (such as meat-to-fat ratio) and appearance (color of skin/meat) beyond promoting growth/weight gain in growing livestock is still a question. Insofar as is known, cysteamine compounds have not been previously reported as being useful for improving agricultural meat production.

### BRIEF SUMMARY OF THE INVENTION

The subject invention provides materials and methods for improving animal agriculture, in particular in the production of meat producing livestock such as poultry, cattle, swine, and sheep. The present invention concerns materials and methods for providing a feed strategy to improve (1) productive efficiency, (2) carcass composition and appearance, and (3) reduce environmental and societal costs associated with livestock wastes.

For example, it is contemplated that the systems and methods of the present invention improve any one or combination of the following: animal reproductive performance (including shortening rebreeding time, increasing conception rates, and increasing litter size); gestational growth and development; newborn growth and development; lactation; overall appearance (such as body shape, meat or skin/fur appearance, and meat to fat ratio); manure production (including decrease the volume of livestock waste produced, reduce odors from manure, as well as reduce the amount of nitrogen excreted in livestock waste); and use of dietary nutrients (including efficient digestion and absorption of dietary nutrients such as vitamins, minerals, and proteins).

According to the present invention, an effective amount of a cysteamine compound is introduced to livestock to promote meat-producing animal health, growth, and population numbers. For example, cysteamine, or various cysteamine salts, prodrugs, analogs, derivatives, conjugates, and metabolites, are administered to cattle, sheep, and swine.

In one embodiment of the invention, a composition comprising a cysteamine compound is introduced into water and/or feed that is subsequently consumed by the livestock animal. In a related embodiment, the composition comprises additional agents that are useful in promoting livestock health. For example, antibiotics and/or vaccines may be concurrently administered with a cysteamine compound to agricultural livestock.

The method and composition of the invention are useful for treating agricultural livestock during any stage of development (including reproductive cycle) to improve health and promote productivity. A cysteamine compound is preferably administered to livestock by introducing the cysteamine compound into water and/or feed.

Preferably, compositions of the present invention comprise 1 to 90 wt % of an effective amount of a cysteamine compound. More preferably, the compositions of the invention comprise 75 wt % of an effective amount of a cysteamine compound. The amount of cysteamine compound used can be varied based upon the health (i.e., size, age, etc.) of the livestock to be treated.

Certain compositions of the invention comprise any one or combination of the following: 5 to 50 wt % of binders and disintegrants; 0.05 to 0.3 wt % of flavoring and/or smelling agents for enhancing the flavor of the composition; enteric coating materials; and additives/agents (such as antibiotics and/or vaccines).

In certain related embodiments, the cysteamine compound of the invention is provided in the form of granules, where the granule comprises at least one or more layers of coating materials. In a preferred embodiment, in each of the granules, the cysteamine compound is shielded from its surroundings by cyclodextrin, or its derivative. In certain embodiments, the granules of cysteamine compound have a size ranging from 0.28 to 0.90 mm in diameter. Such granules may be admixed with livestock animal feed and/or aqueous solutions (such as water or milk) to promote livestock growth and productivity.

### BRIEF DESCRIPTION OF DRAWINGS

FIG. 1 shows a metabolic pathway of cysteamine.

FIG. 2 shows cysteamine as a constituent of co-enzyme A.

FIG. 3 shows a sample sensory evaluation questionnaire and corresponding scoring system in accordance with one embodiment of the invention.

FIG. 4 are comparative illustrations of cuts of meat from swine treated with Cysteamine Hydrochloride versus swine without Cysteamine Hydrochloride treatment.

### DETAILED DISCLOSURE OF THE INVENTION

The subject invention provides unique materials and methods for improving the health and promoting the production of agricultural livestock. Specifically, the subject invention provides materials and methods for improving any one or combination of the following: animal reproductive performance (including shortening rebreeding time, increasing conception rates, and increasing litter size); gestational growth and development; newborn growth and development; lactation; and appearance (such as overall body shape, meat or skin/fur appearance, and meat to fat ratio).

The invention concerns administering a cysteamine compound to agricultural livestock, in an amount effective to promote livestock health, growth, population numbers, and carcass composition and appearance. One embodiment of the invention is a feed or aqueous composition for promoting livestock production, wherein the composition comprises a cysteamine compound. For example, the composition can be an aqueous mixture or an aqueous emulsion including the cysteamine compound. Alternatively, a cysteamine compound (i.e., cysteamine hydrochloride) is provided in solid feed.

According to a first aspect of the present invention, there is provided a method of preparing a composition for promoting animal livestock production comprising the steps of preparing a cysteamine compound, and mixing the cysteamine compound with livestock animal feed and/or aqueous solutions (such as water or milk). In a related embodiment, the cysteamine compound is first mixed with cyclodextrin or its derivative in a reactor and then added to livestock animal feed and/or aqueous solutions (such as water or milk).

The present invention is based on the demonstration that a cysteamine-containing composition when ingested by agricultural livestock animals has activity in improving productive efficiency and carcass composition and appearance. Prior to this finding, there was no suggestion or sufficient indication that administration of the cysteamine-containing composition in livestock animals might have such activity in a large-scale application to effectively and safely: improve animal reproductive performance (including shortening rebreeding time, increasing conception rates, and increasing litter size); promote gestational growth and development; promote newborn growth and development; improve lactation; and enhance overall appearance (such as body shape, meat or fur/skin appearance, and meat to fat ratio).

The present invention also provides a method of manufacture and a use of the cysteamine-containing composition for farming livestock animals by feeding a basal feed (or diet) mixed with the cysteamine compound in order to improve productivity and/or carcass appearance/composition. The invention may be practised by directly mixing the cysteamine compound with a suitable basal feed and/or aqueous solution. Alternatively, the invention may be practiced by mixing firstly a premix made of the cysteamine compound and other ingredients, and secondly the premix with a suitable basal feed and/or aqueous solution to form a final feed/solution. A basal feed is a diet that an animal is normally fed with and an aqueous solution is a solution that is normally administered to an animal. According to the subject invention, different livestock animals will require different basal feed and/or aqueous solution.

It is believed that cysteamine having a physiological activity acts as a growth stimulator. Natural cysteamine is a part of coenzyme A (also know as CoA-SH or CoA) which is a coenzyme pattern of pantothenic acid. In the course of metabolism, coenzyme A acts as the carrier of dihydrosulfuryl or variants of hydrosulfuryl which is linked with the hydrosulfuryl of coenzyme A. Experiments performed on animals such as pigs, poultry, fowls, goats, rabbits and fishes have shown that cysteamine can deplete somatcstatin (SS) in organisms, and in particular in the median eminence nerve terminal and periventricular nuclear neure soma. This increases the level of growth hormone in the blood of the animals which at the same time raises the level of various other growth stimulating factors including insulin-like growth factor I (IGF-I), insulin, triiodothyronine (T3), trthyroxine (T4) and beta-endorphin (beta-END).

With the increase of these various growth promoting factors, the digestive metabolic rate of the animal is correspondingly increased. It is understood that the general protein synthesis rate of the animal is accordingly increased.

The regulation of the physiology by cysteamine is further explained as follows:
(i) Cysteamine can improve somatostatin metabolism and transportation and promote degradation of somatostatin by effecting the vesicles for storing somatostatin.
(ii) Cysteamine can change the structure and conformation of somatostatin by affecting the dimercapto bonds formed at 3- and 14-positions of SS-14 as well as at 17- and 18-positions of SS-28. This is important in regulating the bioactivity and immuno-reactivity of the physiology of the animal.
(iii) Cysteamine can regulate somatostatin receptors, and reduce the affinity of gastric mucosa cell receptors of the animals fed with the cysteamine-containing composition. Cysteamine is an ingredient for forming coenzyme A which can exhaust somatostatin in tissue organs and in the bloodstream of the animals. Cysteamine can also promote synthesis and release of endogenous growth hormone, regulate production of nerve endocritic hormone, enhance basal level, peak value and total level of various growth hormone.

It is also believed that cysteamine can also influence the oestrus cycle in livestock animals as well as improve fertilization. Without being bound by theory, cysteamine appears to increase the efficiency of blastocyst production in immature oocytes, thus decreasing the time for oocyte maturation. Moreover, cysteamine appears to increase male pronuclear development and normal embryonic development. For example, the administration of cysteamine appears to result in a higher proportion of oocytes with synchronously formed male and female pronuclei. Moreover, the administration of cysteamine to livestock animals appears to increase the percentage of cleaving embryos developing to the 8-cell, morula, and blastocyst stage. Finally, the administration of a cysteamine compound to livestock animals appears to improve the quality of livestock animal semen; specifically increasing the number of active and moving spermatoa.

In certain embodiments of the invention, compositions are provided comprising a cysteamine compound and a carrier. Certain compositions of the invention comprise 1 to 95 wt % of cysteamine hydrochloride and 1 to 80 wt % of carrier that is an inclusion host compound material. An inclusion compound host material preferably comprises cyclodextrin and/or its derivative.

According to the subject invention, inclusion compound host materials can be selected from the group consisting of: cyclodextrin and/or its derivative such as, but not limited to, methyl β-cycoldextrin (M-β-CD), hydropropyl β-cycoldextrin (HP-β-CD), hydroethyl β-cycoldextrin (HE-β-CD), polycyclodextrin, ethyl β-cyclodextrin (E-β-CD) and branched cycoldextrin. In a preferred embodiment, a cysteamine compound is mixed with a β-CD form of cyclodextrin because the internal diameter of the β-CD molecule is about 6-8 Å, which makes it a particularly suitable candidate as an inclusion compound host material for preparation of a composition of the invention to be administered to animal livestock.

Mixing of a cysteamine compound of the invention with cyclodextrin, or its derivative, may be performed under the protection of an inert substance. The method for mixing the cysteamine compound with cyclodextrin (or its derivative) may comprise heating the cysteamine compound and the cyclodextrin while mixing for a period of time at a temperature of substantially 25 to 40° C. The method may also comprise stirring the cysteamine or its salt and the cyclodextrin or its derivative to form a first mixture. Preferably, the method may comprise drying the first mixture at a temperature of substantially 40 to 50° C., and this is preferably performed in vacuum. The method may also comprise grounding and/or sieving the first mixture through a mesh screen (e.g. 40-mesh, which means that there are forty pores for each square inch in the mesh screen) to form a second mixture. While a 40-mesh screen may be used, a screen with different mesh size may also be used depending on the size of granules of the composition desired. The second mixture may then be mixed with at least one of fillers, disintegrants and binders to form a third mixture which may be pelleted to form the granules. Thereafter, coating materials made of ingredients selected from a group including cellulose acetate phthalate, polyethylene glycol terephthalate, ethyl acetate and isopropyl acetate may be applied on the granules.

As used herein, the term “livestock” (including the terms “animal livestock,” “agricultural livestock,” or “livestock animal(s)”) refers to varieties of animals raised by man for the purposes of food, leather, or fur production. As contemplated herein, livestock includes, but it not limited to, cattle (milk and beef breeds, bison, buffalo, etc.), swine (including hogs, pigs, and the like), sheep, goats, rabbits, mink, deer, alligator, snakes, and poultry (including chickens, ostrich, turkeys, ducks, geese, quails, game hens, and the like).

It is contemplated that the systems and methods of the present invention improve productivity and/or carcass composition or appearance. This includes improving any one or combination of the following: animal reproductive performance (including shortening rebreeding time, increasing conception rates, and increasing litter size); gestational growth and development; newborn growth and development; lactation; and overall meat appearance and meat to fat ratio.

“Concurrent administration” and “concurrently administering,” as used herein, includes administering a compound or method suitable for use with the methods of the invention (administration of a cysteamine compound) to improve livestock productivity and/or carcass composition/appearance. For example, an antibiotic and/or vaccine can be administered concurrently with the materials and methods of the invention to improve livestock productivity.

According to the subject invention, a compound can be provided in admixture with a cysteamine compound, such as in an aqueous emulsion; or the compound and cysteamine can be provided as separate compounds, such as, for example, separate compositions administered consecutively, simultaneously, or at different times. Preferably, if the cysteamine compound and the known agent (or therapeutic method) for improving livestock productivity and/or carcass composition/appearance are administered separately, they are not administered so distant in time from each other that the cysteamine compound and the known agent (method) cannot interact.

Contemplated compounds that can be concurrently administered with a cysteamine compound of the invention include, but are not limited to, metabolic modifiers (such as bovine somatotropin or porcine somatropin or growth hormone releasing factor); vaccines (such as pseudorabies vaccine, foot-and-mouth vaccine, avian flu vaccine, and vaccines against parasitic disease such as tapeform); and antibodies (such penicillin and tetryacycline).

As used herein, reference to a “cysteamine compound” includes cysteamine, various cysteamine salts (that do not greatly reduce or inhibit the activity of the cysteamine compound), as well as prodrugs of cysteamine that can, for example, be readily metabolized by the livestock to produce cysteamine endogenously. Also included within the scope of the subject invention are analogs, derivatives, conjugates, and metabolites of cysteamine, which have the ability as, described herein to improve livestock health. Various analogs, derivatives, conjugates, and metabolites of cysteamine are well known and readily used by those skilled in the art and include, for example, compounds, compositions and methods of delivery as set forth in U.S. Pat. Nos. 6,521,266; 6,468,522; 5,714,519; and 5,554,655.

As contemplated herein, a cysteamine compound includes pantothenic acid. Pantothenic acid is a naturally occurring vitamin that is converted in mammals to coenzyme A, a substance vital to many mammalian physiological reactions. Cysteamine is a component of coenzyme A, and increasing coenzyme A levels results in increased levels of circulating cysteamine. Alkali metal salts, such as magnesium phosphate tribasic and magnesium sulphite (Epsom salts), enhance formation of coenzyme A. Furthermore, breakdown of coenzyme A to cysteamine is enhanced by the presence of a reducing agent, such as citric acid. Thus, the combination of pantothenic acid and alkali metal salts results in increased coenzyme A production and, concomitantly, cysteamine.

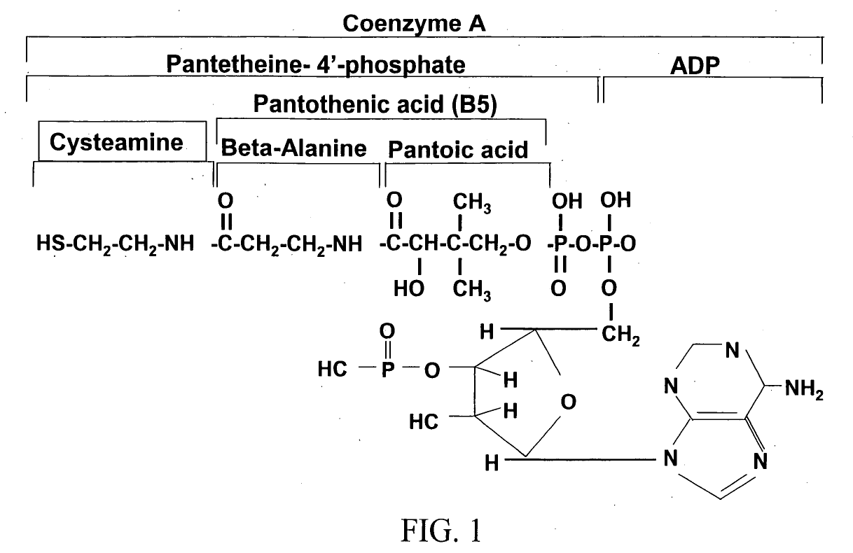

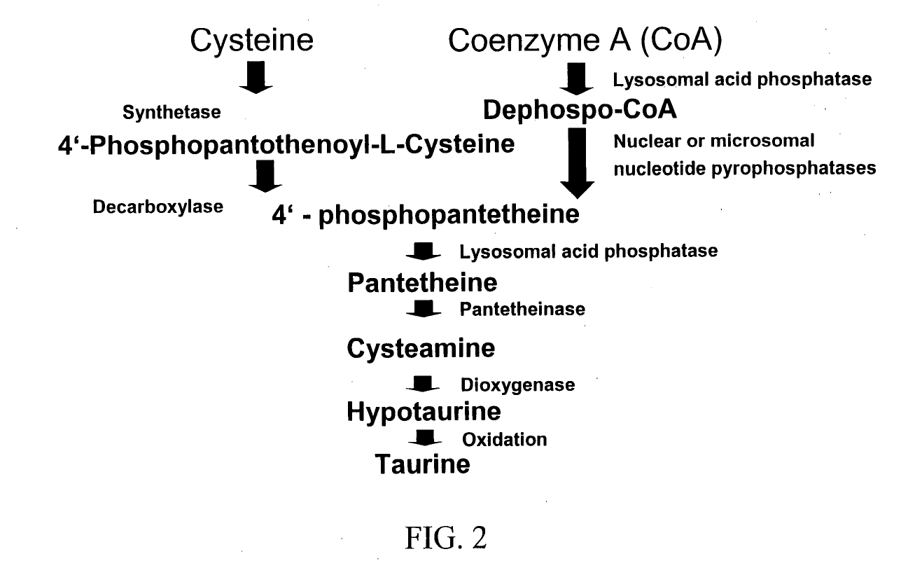

The advantages of cysteamine, as set forth herein, may be achieved in livestock by promoting the endogenous production of cysteamine through natural metabolic processes (i.e., through the action of co-enzyme A or as a metabolite of cysteine (see FIGS. 1 and 2 of mammalian production of cysteamine)). This may be achieved by, for example, the administration of pantothenic acid to livestock.

The term “effective amount,” as used herein, refers to the amount necessary to elicit the desired biological response. In accordance with the subject invention, the effective amount of a cysteamine compound is the amount necessary to improve livestock productivity and/or carcass composition/appearance. In a preferred embodiment, the effective amount of a cysteamine compound is the amount necessary to improve livestock reproductive performance (including shortening rebreeding time, increasing conception rates, and increasing litter size); promote gestational growth and development; promote newborn growth and development; improve livestock lactation; and/or enhance overall meat appearance and meat to fat ratio. For example, the improvement in livestock productivity and/or carcass composition/appearance can be a 1%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, or 300% acceleration and/or augmentation in growth; improvement in immunity response to a disease and/or contaminant; and/or enhancement in fertility.

Accelerated and/or Augmented Growth

In one embodiment of the invention, a cysteamine compound is administered to livestock to accelerate and/or augment growth. As contemplated herein, to “accelerate and/or augment growth” refers to the ability to shorten developmental periods during a normal growth cycle and/or increase the overall size of the livestock.

For example, with swine (such as pigs), the materials and methods of the invention could shorten the length of time by five to ten days from a normal growth cycle to reach marketable size. Or, the invention can increase the overall size of a pig from about 3-7 kg from the application stage of 30 to 85 kg body weight of swine. In certain embodiments, the subject materials and methods of the invention both accelerate and augment growth (i.e., decrease the length of normal growth cycle and increase overall size of the livestock).

Enhancing Fertility

According to the subject invention, a cysteamine compound is administered to livestock to enhance fertility. As contemplated herein, to “enhance fertility” refers to the ability to maximize fertilization and reduce oestrus cycle of livestock. In one embodiment of the subject invention, a cysteamine compound is administered to livestock to manipulate sexual development. Sexual development manipulation can include increasing the number of eggs and sperm that are produced and discharged by livestock or shortening the time to which livestock are capable of reproduction.

Enhancing Appearance

As noted earlier, several deficiencies exist in present livestock breeding methods. For example, livestock raised in narrow, confined sheds prevent their meat and skin from achieving fine texture, firmness, tenderness, and tastiness (and, in the case of skin/fur, high quality). Due to these deficiencies, the meat and skin of livestock become tough, coarse and fatty, and generally, they do not meet with public favor as food or leather products. The subject invention enables qualitative improvement of livestock meat and skin by administering a cysteamine compound to livestock. In a preferred embodiment, 150 ppm (parts per million) of cysteamine hydrochloride is added to livestock feed to enhance livestock appearance.

Specifically, the subject invention concerns a method for producing fine, firm, tender, and tasty (and, in the case of skin/fur, high quality) meats and skins of livestock. By this invention, it is possible to raise livestock with good body shape, superior meat appearance as well as desirable meat to fat ratio to provide the consumer with food products having such traits as fine texture, firmness, tenderness, and tastiness. More importantly, in the case of skin/fur, the invention enables production of livestock with good complexion (such as firmness, suppleness, etc.) and hair/fur coat (such as fine texture, shine, etc.) to provide the consumer with fur/leather products of high quality.

Administration of a cysteamine compound to livestock, in accordance with the subject invention, can be accomplished by any suitable method and technique presently or prospectively known to those skilled in the art. Specifically exemplified herein is the introduction of a cysteamine compound, either alone or concurrently with additional compound(s) or method(s), into feed and/or aqueous solutions to be administered to livestock. The cysteamine compound can be introduced as a composition, in any available form including in a liquid (i.e., solvent, oil), in an aqueous mixture, in an aqueous emulsion, in a solid carrier or substrate, or other vehicles provided the vehicles are compatible with the administration of the cysteamine compound into solid feed and or aqueous solution, and do not adversely affect the livestock.

A variety of suitable adjuvants may also be used in compositions comprising a cysteamine compound. For example, emulsifiers, antifoaming agents (or defoaming agents), antioxidants, preservatives, coloring agents, and the like can be included in compositions of the invention. In one embodiment, the adjuvants are present in compositions of the invention in minor amounts, i.e., less than about 5% by volume, and preferably, less than 1% by volume. In other embodiments, greater amounts of adjuvants are present in compositions of the invention, i.e., up to 70% by volume. All such adjuvants should be noninjurious and nontoxic to livestock being treated.

According to the present invention, suitable emulsifiers (i.e., surfactants or dispersants) can be cationic, anionic, nonionic, or amphoteric emulsifiers. Preferred emulsifiers include, for example, food grade emulsifiers which are widely available. An overview of some types of suitable emulsifiers for use with the invention include those set forth in A. J. St. Angelo, “A Brief Introduction to Food Emulsion and Emulsifiers,” at pp. 1-8 of G. Charalambous et al., Eds., Food Emulsifiers—Chemistry, Technology, Functional Properties and Applications, Elsevier Science Publishing Co. Inc., New York, N.Y. (1989).

Where needed, after the introduction of a cysteamine compound to feed products and/or aqueous solutions for administration to livestock to be treated, a metering or mixing pump, or an inline mixer (i.e., a mixing valve, nozzle or orifice), an aerator, or other device known to the skilled artisan may be used to accomplish the direct dispersion of the cysteamine compound in feed and/or aqueous solution.

In a preferred embodiment, a solid feed mixture including a cysteamine compound is introduced to livestock to be treated. The feed mixture of the invention can contain from about 0.1% to about 95% of a cysteamine compound, wherein all percentages being by volume, based on the final volume of the composition. The amount of cysteamine compound used can be varied based upon the health (i.e., size, age, etc.) of the livestock to be treated.

The cysteamine compounds of the subject invention can be formulated according to known methods for preparing compositions for use in administration to livestock. The formulations may be presented in unit-dose or multi-dose containers, for example sealed ampoules and vials, and may be stored in a freeze dried (lyophilized) condition requiring only the condition of the sterile liquid carrier, for example, water, prior to use. Extemporaneous solutions and suspensions may be prepared from sterile powder, granules, tablets, etc. It should be understood that in addition to the ingredients particularly mentioned above, the formulations of the subject invention can include other agents conventional in the art having regard to the type of formulation in question.

Animal Feed Additives

Apart from the cysteamine compound, the animal feed additives of the invention contain at least one fat-soluble vitamin, and/or at least one water-soluble vitamin, and/or at least one trace mineral, and/or at least one macro mineral.

Further, optional, feed-additive ingredients are colouring agents, aroma compounds, stabilisers, and/or at least one other enzyme selected from amongst phytases EC 3.1.3.8 or 3.1.3.26; xylanases EC 3.2.1.8; galactanases EC 3.2.1.89; and/or beta-glucanases EC 3.2.1.4 (EC refers to Enzyme Classes according to Enzyme Nomenclature 1992 from NC-IUBMB, 1992).

Usually fat- and water-soluble vitamins, as well as trace minerals form part of a so-called premix intended for addition to the feed, whereas macro minerals are usually separately added to the feed. Either of these composition types, when enriched with a cysteamine compound according to the invention, is an animal feed additive of the invention.

In a particular embodiment, the animal feed additive of the invention is intended for being included (or prescribed as having to be included) in animal diets or feed at levels of 0.01-10.0% in combination with a cysteamine compound; more particularly 0.05-5.0%; or 0.2-1.0% (% meaning g additive per 100 g feed). This is so in particular for premixes.

Accordingly, the concentrations of the individual components of the animal feed additive, e.g. the premix, can be found by multiplying the final in-feed concentration of the same component by, respectively, 10-10000; 20-2000; or 100-500 (referring to the above three percentage inclusion intervals).

Guidelines for desired final concentrations, i.e. in-feed-concentrations, of such individual feed and feed additive components are indicated in Table 1 below.

The following are non-exclusive lists of examples of these components. Examples of fat-soluble vitamins are vitamin A, vitamin D3, vitamin E, and vitamin K, e.g. vitamin K3.

Examples of water-soluble vitamins are vitamin B12, biotin and choline, vitamin B 1, vitamin B2, vitamin B6, niacin, folic acid and panthothenate, e.g. Ca-D-panthothenate.

Examples of trace minerals are manganese, zinc, iron, copper, iodine, selenium, and cobalt.

Examples of macro minerals are calcium, phosphorus and sodium.

The nutritional requirements of these components—exemplified with poultry and piglets/pigs—are listed in Table 1 below. Nutritional requirement means that these components should be provided in the diet in the concentrations indicated. These data are compiled from: NRC, Nutrient requirements in swine, tenth revised edition 1998, subcommittee on swine nutrition, committee on animal nutrition, board on agriculture, national research council. National Academy Press, Washington, D.C. 1998; and NRC, Nutrient requirements of poultry, ninth revised edition 1994, subcommittee on poultry nutrition, committee on animal nutrition, board of agriculture, national research council. National Academy Press, Washington, D.C. 1994.

In the alternative, the animal feed additive of the invention comprises a cysteamine compound and at least one of the individual components specified in Table 1. At least one means either of, one or more of, one, or two, or three, or four and so forth up to all thirteen, or up to all fifteen individual components.

More specifically, this at least one individual component is included in the additive of the invention in such an amount as to provide an in-feed-concentration within the range indicated in column four, or column five, or column six of Table 1.

As explained above, corresponding feed additive concentrations can be found by multiplying the interval limits of these ranges with 10-10000; 20-2000; or 100-500. As an example, considering which premix-content of vitamin A would correspond to the feed-content of 10-10000 IU/kg, this exercise would lead to the following intervals: 100-10⁸ IU; or 200-2×10⁷ IU; or 1000-5×10⁶ IU per kg additive.

TABLE 1
Nutrient Requirements and Preferred Ranges

| | | | | | |
|---|---|---|---|---|---|
| Nutrients | | | | | |
| provided per kg | | Piglets, | | | |
| diet | Poultry | Pigs, Sows | Range 1 | Range 2 | Range 3 |

Fat Soluble Vitamins

| | | | | | |
|---|---|---|---|---|---|
| Vitamin A/[IU] | −5000 | 1300-4000 | 10-10000 | 50-8000 | 100-6000 |
| Vitamin D₃/[IU] | −1100 | 150-200 | 2-3000 | 5-2000 | 10-1500 |
| Vitamin E/[IU] | −12 | 11-22 | 0.02-100 | 0.2-80 | 0.5-50 |
| Vitamin K/[mg] | 0.5-1.5 | −0.5 | 0.005-10.0 | 0.05-5.0 | 0.1-3.0 |

Water Soluble Vitamins

| | | | | | |
|---|---|---|---|---|---|
| Vitamin | −0.003 | 0.005-0.02 | 0.0001-1.000 | 0.0005-0.500 | 0.001-0.100 |
| B₁₂/[mg] | | | | | |
| Biotin/[mg] | 0.100-0.25 | 0.05-0.08 | 0.001-10.00 | 0.005-5.00 | 0.01-1.00 |
| Choline/[mg] | 800-1600 | 300-600 | 1-10000 | 5-5000 | 10-3000 |

Trace Minerals

| | | | | | |
|---|---|---|---|---|---|
| Manganese/[mg] | −60 | 2.0-4.0 | 0.1-1000 | 0.5-500 | 1.0-100 |
| Zinc/[mg] | 40-70 | 50-100 | 1-1000 | 5-500 | 10-300 |
| Iron/[mg] | 50-80 | 40-100 | 1-1000 | 5-500 | 10-300 |
| Copper/[mg] | 6-8 | 3.0-6.0 | 0.1-1000 | 0.5-100 | 1.0-25 |
| Iodine/[mg] | −0.4 | −0.14 | 0.01-100 | 0.05-10 | 0.1-1.0 |
| Selenium/[mg] | −0.2 | 0.10-0.30 | 0.005-100 | 0.01-10.0 | 0.05-1.0 |

Macro Minerals

| | | | | | |
|---|---|---|---|---|---|
| Calcium/[g] | 8-40 | 5-9 | 0.1-200 | 0.5-150 | 1-100 |
| Phosphorus, also | 3-6 | 1.5-6 | 0.1-200 | 0.5-150 | 1-50 |
| available as | | | | | |
| phosporus/[g] | | | | | |

Animal Feed Compositions

Animal feed compositions or diets have a relatively high content of protein. According to the National Research Council (NRC) publications referred to above, poultry and pig diets can be characterised as indicated in Table 2 below, columns 2-3.

An animal feed composition according to the invention has a crude protein content of 50-800 g/kg, and furthermore comprises at least one cysteamine compound as claimed herein.

Furthermore, or in the alternative (to the crude protein content indicated above), the animal feed composition of the invention has a content of metabolisable energy of 10-30 MJ/kg; and/or a content of calcium of 0.1-200 g/kg; and/or a content of available phosphorus of 0.1-200 g/kg; and/or a content of methionine of 0.1-100 g/kg; and/or a content of methionine plus cysteine of 0.1-150 g/kg; and/or a content of lysine of 0.5-50 g/kg.

In particular embodiments, the content of metabolisable energy, crude protein, calcium, phosphorus, methionine, methionine plus cysteine, and/or lysine is within any one of ranges 2, 3, 4 or 5 in Table 2 below (R. 2-5).

Crude protein is calculated as nitrogen (N) multiplied by a factor 6.25, i.e. Crude protein (g/kg)=N (g/kg).times.6.25 as stated in Animal Nutrition, 4th edition, Chapter 13 (Eds. P. McDonald, R. A. Edwards and J. F. D. Greenhalgh, Longman Scientific and Technical, 1988, ISBN 0-582-40903-9). The nitrogen content is determined by the Kjeldahl method (A.O.A.C., 1984, Official Methods of Analysis 14th ed., Association of Official Analytical Chemists, Washington D.C.).

Metabolisable energy can be calculated on the basis of the NRC publication Nutrient Requirements of Swine (1988) pp. 2-6, and the European Table of Energy Values for Poultry Feed-stuffs, Spelderholt centre for poultry research and extension, 7361 DA Beekbergen, The Netherlands. Grafisch bedrijf Ponsen & Iooijen bv, Wageningen. ISBN 90-71463-12-5.

The dietary content of calcium, available phosphorus and amino acids in complete animal diets is calculated on the basis of feed tables such as Veevoedertabel 1997, gegevens over chemische samenstelling, verteerbaarheid en voederwaarde van voedermiddelen, Central Veevoederbureau, Runderweg 6, 8219 pk Lelystad. ISBN 90-72839-13-7.

In a particular embodiment, the animal feed composition of the invention contains a cysteamine compound and at least one vegetable protein or protein source as defined above.

In still further particular embodiments, the animal feed composition of the invention contains a cysteamine compound, 0-80% maize; and/or 0-80% sorghum; and/or 0-70% wheat; and/or 0-70% Barley; and/or 0-30% oats; and/or 0-40% soybean meal; and/or 0-20% whey.

Animal diets can e.g. be manufactured as mash feed (non-pelleted) or pelleted feed. Typically, the milled feed-stuffs are mixed and sufficient amounts of essential vitamins and minerals are added according to the specifications for the species in question.

The cysteamine should be applied in an effective amount, i.e. in an amount adequate for improving nutritional value of feed. Preferably, an effective amount of cysteamine compound adequate for improving nutritional value of feed is 150 ppm of cysteamine hydrochloride.

Cysteamine compounds can be added as solid or liquid formulations. For example, a solid cysteamine formulation is typically added before or during the mixing step; and a liquid cysteamine compound preparation is typically added after the pelleting step. The cysteamine may also be incorporated in a feed additive or premix. The final cysteamine compound concentration in the diet is within the range of 0.1-300 g per ton diet, for example in the range of 54-270 g per kg animal diet. Preferably, the final cysteamine compound concentration in the diet is within the range of about 125-175 g per ton diet, even more preferably 150 g per ton of diet.

TABLE 2
Range values for energy, protein and minerals in animal diets

| | | Piglets/Pigs/ | | | | | |
|---|---|---|---|---|---|---|---|
| | Poultry | Sows | | | | | |
| Nutrient | Min-Max | Min-Max | Range 1 | Range 2 | Range 3 | Range 4 | Range 5 |
| Metabolisable | 12.1-13.4 | 12.9-13.5 | 10-30 | 11-28 | 11-26 | 12-25 | — |
| energy, | | | | | | | |
| MJ/kg | | | | | | | |
| Crude | 124-280 | 120-240 | 50-800 | 75-700 | 100-600 | 110-500 | 120-490 |
| Protein, g/kg | | | | | | | |
| Calcium, | 8-40 | 5-9 | 0.1-200 | 0.5-150 | 1-100 | 4-50 | — |
| g/kg | | | | | | | |
| Available | 2.1-6.0 | 1.5-5.5 | 0.1-200 | 0.5-150 | 1-100 | 1-50 | 1-25 |
| Phosphorus, | | | | | | | |
| g/kg | | | | | | | |
| Methionine, | 3.2-5.5 | — | 0.1-100 | 0.5-75 | 1-50 | 1-30 | — |
| g/kg | | | | | | | |
| Methionine + Cysteine, | 4-9 | 2.3-6.8 | 0.1-150 | 0.5-125 | 1-80 | — | — |
| g/kg | | | | | | | |
| Lysine, | 2.5-11 | 6-14 | 0.5-50 | 0.5-40 | 1-30 | — | — |
| g/kg | | | | | | | |

Following are examples that illustrate procedures for practicing the invention. This example should not be construed as limiting. All percentages are by weight and all solvent mixture proportions are by volume unless otherwise noted.

### EXAMPLE 1

### Enhanced Productivity in Swine

The following example was performed to demonstrate the effect on pregnant pig sows when fed a cysteamine compound that is described in greater detail below. A total of 14 heads of pregnant pig sow (which were the offspring of Duroc×Landrace×Meishan swine, also referred to herein as D×L×M hybrid swine) (84 days of pregnant) were divided into 2 groups: Control group (6) and Cysteamine group (8). The Control group was fed with basal diet. The Cysteamine group was fed with basal diet containing 150 g cysteamine hydrochloride per ton feed. After 49 days of the trial, the results illustrated in Table 3 below demonstrate that the Cysteamine group had an increase of 13.28% in litter weight, 12.43% increase in piglet born weight and 8.00% increase in weaning weight.

TABLE 3
Effect of Cysteamine Hydrochloride on Swine Productivity

| | | | | Improvement |
|---|---|---|---|---|
| Items | Control | Cysteamine | Improvement | (%) |
| Animal | Late phase pregnant sow | | | |
| Dosage (g/T) | 0 | 500 | — | — |
| Population (head) | 6 | 8 | — | — |
| Trial period (day) | From 84 days of pregnant to weaning | | | |
| Total piglet born (head) | 61 | 82 | — | — |
| Piglet born per litter (head) | 10.20 | 10.29 | — | — |
| Total live piglet born (head) | 61 | 82 | — | — |
| Live piglet born per litter (head) | 10.20 | 10.29 | — | — |
| Live piglet born rate (%) | 100.00% | 100.00% | 0.00% | 0.00% |
| Birth weight per litter (kg) | 18.82 | 21.32 | 2.50 | 13.28% |
| Birth weight per piglet (kg/head) | 1.85 | 2.08 | 0.23 | 12.43% |
| Piglets weaning age (days) | 19 | 19 | — | — |
| Body weight of weaned piglet (kg/head) | 5.75 | 6.21 | 0.46 | 8.00% |

### EXAMPLE 2

### Enhanced Growth in Swine

The following example was performed to demonstrate the effect on weaning piglets when fed a cysteamine compound that is described in greater detail below. Total of 100 heads of unhealthy D×L×M weanling piglets (approx. 6.78 kg) were divided into 2 groups—Control group (50 heads) & Cysteamine group (50 heads). The Control group was fed with basal diet. The Cysteamine group was fed with basal diet containing 90 g of cysteamine hydrochloride per ton of feed. As illustrated in Table 4 below, after 30 days of the trial, the results showed that the Cysteamine group had an increase of 12.83% in average daily weight gain (ADG), a decrease of 5.56% in feed conversion rate (FCR) and 43.24% improvement in diarrhea condition.

TABLE 4
Effect of Cysteamine Hydrochloride on Weaning Piglets

| | | | | Improvement |
|---|---|---|---|---|
| Item | Control | Cysteamine | Improvement | (%) |
| Animal | DxLxM weanling piglets | | | |
| Treatment | 300 ppm | | | |
| Population (head) | 50 | 50 | | — |
| Trial days | 30 | 30 | | — |
| Initial Wt. (kg/head) | 6.75 | 6.8 | | — |
| Final Wt. (kg/head) | 17.96 | 19.46 | +1.5 | — |
| Total final weight | 898 | 973 | +75 | |
| (kg) | | | | |
| Weight gain (kg/ | 11.21 | 12.66 | +1.45 | — |
| head) | | | | |
| ADG (g/head) | 374 | 422 | +48 | 12.83% |
| Total weight gain | 560.5 | 633 | +72.5 | — |
| (kg) | | | | |
| Daily feed | 673.2 | 717.4 | +44.2 | |
| consumption | | | | |
| (g/head) | | | | |
| Total feed | 1009.8 | 1076.1 | +66.3 | — |
| consumption (kg) | | | | |
| FCR | 1.80 | 1.70 | −0.1 | −5.56% |
| cysteamine | | 96.85 | | |
| hydrochloride | | | | |
| (g) | | | | |
| Diarrhea (head) | 37 | 21 | | −43.24% |

### EXAMPLE 3

### Carcass Composition/Appearance in Swine

The following example was performed to demonstrate the effect on pig carcass composition and appearance after being fed a cysteamine compound that is described in greater detail below. 72 piglets were divided evenly into control group and Cysteamine group. The control group was treated with basal diet while the Cysteamine group was treated with basal diet added with 150 g cysteamine hydrochloride per ton of feed. Upon reaching the market weight of 85-90 kg, five animals from each treatment were slaughtered after fasting for one day. During slaughter, the animals were stunned with a stunning gun, bled, dehaired and eviscerated. The carcass was split into minus the head and then chilled for 24 hours prior to fabrication. The slaughter and the chilled carcass weights were recorded. The chilled carcass weight expressed as percentage of the slaughter weight represents the dressed yield. The wholesale and retail cuts of each animals were weighed and recorded. The values were expressed as the percentage of the live weight and of the chilled carcass weight.

The area of the longissimus dorsi (LD) muscle was measured on the 12^{th} rib of the right part of the carcasses. This was done by tracing the outline of the whole LD muscle on acetate paper and the area was measured with the use of a grading method.

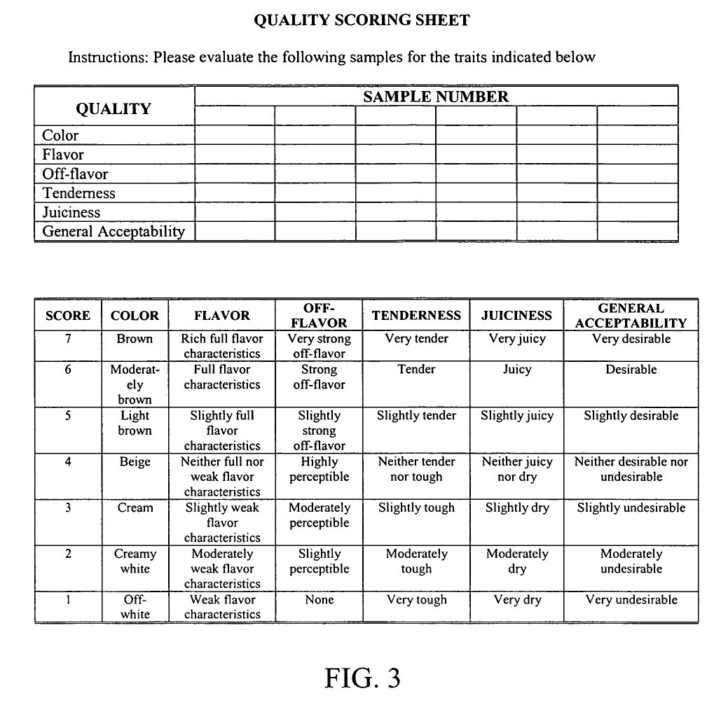

Samples of the loin eye area from each animal were taken and roasted for sensory evaluation by members of an experienced sensory panel. The sensory evaluation (see FIG. 3) constituted a sensory review of meat flavor and various attributes. There was no significant difference observed among preparation of meat taken from swine treated with cysteamine hydrochloride and swine treated without cysteamine hydrochloride. Evaluation members noted that pigs fed with cysteamine hydrochloride had attributes similar to those fed without cysteamine hydrochloride (see Table 5 below).

TABLE 5
Sensory Evaluation of pork from swine fed diets with Cysteamine
HCl and without Cysteamine HCl

| Meat Attribute | With cysteamine HCl | Without cysteamine HCl |
|---|---|---|
| Color | 3.51 | 3.72 |
| Flavor | 5.15 | 5.35 |
| Off-Flavor | 1.56 | 1.55 |
| Tenderness | 4.69 | 4.9 |
| Juiciness | 3.92 | 4.20 |
| General Acceptability | 4.70 | 4.95 |

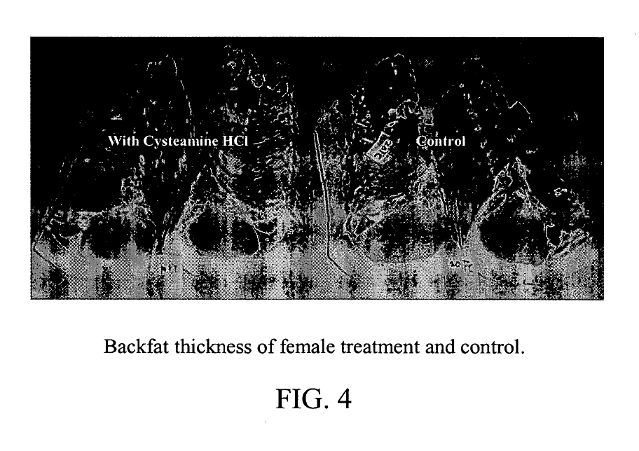

Table 6 below shows the carcass characteristics of fattening pigs from the different treatments as described above. There were no significant differences in dressing percentage, loin eye area, and lean:fat ratio. There were no significant interaction effects. However, pigs fed diets with cysteamine hydrochloride yielded significantly thinner backfat than those from pigs fed diet without cysteamine hydrochloride (see FIG. 4). Likewise, the loin cut and lean trimmings as a percent of the live-weight from pigs treated with cysteamine hydrochloride were significantly higher than those fed diets without cysteamine hydrochloride. These results indicate that cysteamine hydrochloride tends to improve muscle tissue growth in pigs receiving cysteamine hydrochloride.

TABLE 6
Effect of Cysteamine Hydrochloride on Carcass Composition/
Appearance

| | | Loin Eye Area | Lean:Fat | Backfat |
|---|---|---|---|---|
| | Dressing % | (sq.cm) | Ratio | Thickness (cm) |
| Cysteamine | 69.012 | 4.850 | 0.105 | 1.91^{a} |
| HCl | | | | |
| Non- | 67.950 | 4.750 | 0.101 | 2.16^{b} |
| Cysteamine | | | | |
| HCl | | | | |

^{a}and ^{b}represent statistically significant differences (P < 0.05) between the two samples.

### EXAMPLE 4

### Increase in Intestine Villi Length

The following example was performed to demonstrate the effect on pig intestine villi length after being fed a cysteamine compound that is described in greater detail below. 72 piglets were evenly divided into control group and Cysteamine group. The control group was treated with basal diet only, while the Cysteamine group was added with 150 g of cysteamine hydrochloride per ton of feed. The trial was lasted for 30 days.

Immediately after evisceration, tissue samples from the small intestine, large intestine, liver, and pancreas of pigs from each treatment group were collected. The collected samples were placed in properly labeled containers containing 10% buffered neutral formalin solution. The tissue samples were fixed for 78 hours before they were processed for routine histological examination using the paraffin embedding technique (well-known to the skilled artisan).

Sections approximately 5 to 6 μ thick were cut from paraffin blocked tissues using Optical 820 Rotary Microtome (Scientific Instruments Division, Buffalo, N.Y.). Some sections were stained with hematoxylin and eosin (H & E), others with Alcian blue. H & E stained sections were used for the examination of the general histology of the intestinal tract while the Alcian blue stained sections were used for goblet cells identification. The stained sections were examined under various magnifications using a light microscope.

The number and length (μm) of villi in the small intestine were counted per microscopic field; the thickness of the tunica muscularis in the small and large intestine were measured using American Optical Micrometer (American Optical Company, Buffalo, N.Y.) calibrated in millimeters. The mucosal folds in the large intestine, the density of intestinal glands (crypts), the appearance of the surface epithelium were all observed. The general histology of the liver and pancreas were examined and compared between Cysteamine treated and non-Cysteamine treated groups. Goblet cell density in the Cysteamine treated and non-Cysteamine treated pigs was compared through visual examination using light microscope.

The number and length of small intestinal villi were more numerous and longer in Cysteamine treated than in non-Cysteamine treated groups (Table 7).

TABLE 7
Effect of Cysteamine on Villi Length

| Treatment Group | Small Intestines |
|---|---|
| Non-Cysteamine | 1.88 ± 0.14^{A} |
| Cysteamine | 2.15 ± 0.09^{A} |

In Table 7, the same alphabet letters in the same line indicate that the result differences are not statistically significant (p>0.05); the different alphabets in the same line indicate the result differences are statistically significant (p<0.05).

In addition, the goblet cells in the small and large intestine in the Cysteamine-treated livestock were noticeably larger and highly distended than those in non-Cysteamine-treated livestock. Normally, goblet cells have a typical glass wine appearance.

It is hypothesized that with the increased number and length of small intestinal villi observed with Cysteamine-treated livestock, there is a decrease in livestock waste volume, including a decrease in amount of nitrogen excreted in livestock waste, especially in pigs and poultry. Nitrogen can contaminate ground and surface waters, contribute to “acid rain,” which increases the acids in soils and be the source of unwanted odors.

Further, it is hypothesized that with the increased number and length of small intestine villi observed with Cysteamine-treated livestock, there is an increased efficiency in nutrient usage by the livestock. For example, with increased efficiency in the use of amino acids such as lysine, methionine, tryptophan, threonine, and other essential amino acids in dietary feed, the nutritional requirements of livestock (such as pigs and poultry) can be met with lower protein diets. Such diets contain fewer excesses of other amino acids that eventually must be degraded to urea nitrogen and excreted in the urine. It is expected that feeding livestock a cysteamine compound of the invention greatly reduces the amount of nitrogen (such as urea) from being excreted into the environment.

### EXAMPLE 5

### Enhanced Productivity in Beef Cattle

In the following example, the test animals were beef cattle (Ximenta'er hybridizing beef cattle). The experiment was performed at the Hebei Hengshui Geling Beef Cattle Raising Farm dur the period from Oct. 10 to Dec. 21, 2002. In total, 64 heads of around 350 kg Ximenta'er hybridizing beef cattle were selected and separated into 4 groups randomly according to the principle of equalizing age, weight, grace, fur/hair condition, etc. Each group had 16 heads of beef cattle. The Control Group, Test Group I, II & III were fed a composition comprising 0, 1.5, 3, 6 g/head/day of cysteamine HCl, respectively. As noted in Tables 8, 9, and 10 below, adding a cysteamine compound to the basal diet of beef cattle can increase daily weight gain.

TABLE 8
Effect of Cysteamine HCl on Daily Weight Gain (kg) of Beef Cattle

| Case | Control | Test I | Test II | Test III |
|---|---|---|---|---|
| Initial Wt. (kg) | 356.3 | 356.7 | | 356.0 |
| Final Wt. (kg) | 422.0 | 423.8 | 420.5 | 432.7 |
| Daily weight gain (g) | 1.05^{a} | 1.065^{a} | 1.025^{a} | 1.217^{b} |

TABLE 9
Effect of Cysteamine HCl on Feed Conversion Rate (FCR) of
Beef Cattle

| Case | Control | Test I | Test II | Test III |
|---|---|---|---|---|
| Concentrated feed | 3.70^{a} | 3.54 | 3.70^{a} | 3.09^{b} |
| Raw Grass | 1.27^{a} | 1.21 | 1.27^{a} | 1.06^{a} |
| Corn Silage | 15.42^{a} | 14.14 | 14.42^{a} | 12.87^{b} |

In Tables 8 and 9, the same alphabets in the same line indicate that the result differences are not statistically significant (p>0.05); the different alphabets in the same line indicate the result differences are statistically significant (p<0.05).

TABLE 10
Fattening Beef Cattle Concentrated Feed Components

| Concentrated feed components (%) | Initial Phase | Late Phase |
|---|---|---|
| Corn grain | 48.00 | 53.00 |
| Wheat noodle | 20.00 | 17.00 |
| Soybean meal | 27.00 | 25.00 |
| Zeolite | 3.00 | 3.00 |
| Salt | 1.00 | 1.00 |
| Sodium Bicarbonate | 1.00 | 1.00 |
| Total | 100.00 | 100.00 |

The results illustrated in Tables 8, 9, and 10 indicate that adding cysteamine HCl into the basal diet of Ximenta'er hybridizing beef cattle can increase the daily weight gain. Specifically, adding 6 g/head/day of cysteamine HCl in Test Group III had an increase of 1.217 kg in daily weight gain, with significant differences (p<0.05) result better than the Control Group, Test Group I & II. Thus, according to one embodiment of the invention, the appropriate additive volume of a cysteamine compound to be administered to livestock is 6 g/head/day. Cysteamine HCl is also good for increasing beef cattle productivity due to its suitable taste quality. Moreover, adding cysteamine HCl to beef cattle diet can increase their immunity to various diseases and conditions.

### EXAMPLE 6

### Enhanced Productivity in Sheep Livestock

In the following example, the test animals were sheep (small-fat-tail sheep). The experiment was performed at the Goat Trial Farm of Beijing Shengshifumin Muslim Food Company during the thirty-five day period from Dec. 2, 2004 to Jan. 5, 2005. In total, 40 heads of small-fat-tail sheep with the average weight of 26.5 kg were randomly divided into Control group (20) and Cysteamine group (20). The control group was fed with basal diet only, while the Cysteamine group was fed a diet that included 900 g cysteamine HCl per tonnage of diet. The standard basal diet of the farm was employed for this trial. As illustrated in Tables 11 and 12, after 35 days of adding cysteamine HCl to the basal diet, a statistically significant increase in weight in both the small-fat-tail sheep and crossbreed sheep ADG was observed a well as a reduction in the amount of silage necessary in raising the sheep.

TABLE 11
Effect of Cysteamine Compound on Small-Fat-Tail Sheep

| Item | Control | Cysteamine HCl | Improvement (%) |
|---|---|---|---|
| Dosage (g/tonnage) | | 900 | |
| Age (day) | 135 | 135 | |
| Trial Period (day) | 35 | 35 | — |
| Population (head) | 20 | 20 | — |
| Initial weight | 26.82 | 25.57 | — |
| (kg/head) | | | |
| Final weight (kg/head) | 29.38 | 28.92 | — |
| Weight gain (kg/head) | 2.560 | 3.350 | 30.9% |
| Daily weight gain | 73.14 | 95.71 | 30.9% |
| (g/head/day) | | | |
| Concentrate (kg) | 245 | 245 | — |
| Silage (kg) | 506.55 | 424.38 | −16.22% |
| Hay (kg) | 350.00 | 350.00 | — |
| Total feed | 1101.55 | 1019.38 | — |
| consumption (kg) | | | |

TABLE 12
Effect of Cysteamine Compound on Crossbreed Sheep

| Item | Control | Cysteamine HCl | Improvement (%) |
|---|---|---|---|
| Dosage (g/tonnage) | | 900 | |
| Age (day) | 135 | | |
| Trial period (day) | 35 | 35 | — |
| Population (head) | 20 | 20 | — |
| Initial weight (kg/head) | 26.92 | 26.71 | — |
| Final weight (kg/head) | 28.89 | 29.59 | — |
| Weight gain (kg/head) | 1.97 | 2.88 | 46.2% |
| ADG (g/head/day) | 56.29 | 82.29 | 46.2% |
| Concentrate (kg) | 245 | 245 | — |
| Silage (kg) | 428.2 | 406.2 | −5.14% |
| Hay (kg) | 350.0 | 350.0 | — |
| Total feed consumption | 1023.2 | 1001.2 | — |
| (kg) | | | |

### EXAMPLE 7

### Swine Feeds

According to one embodiment of the invention, a cysteamine compound (such as 90 g of cysteamine HCl per tonnage of feed) is added to a basal feed diet that is given to weanling pigs. Based feed diet fed to weanling pigs (also referred to herein as starter diet) preferably are formulated with a cysteamine compound of the invention and includes a crude protein level of about 18% and 3200 kcal ME.

An example of those materials that make up a basal feed diet for weanling pigs is provided in Table 13 below. The nutrient content that make up a basal feed diet for weanling pigs is provided in Table 14 below.

TABLE 13
Composition of pre-starter diet for weanling swine

| Ingredients | % of Feed Content |
|---|---|
| Corn, yellow | 60.500-61.600 |
| Soybean meal, 46% | 26.400-29.100 |
| Rice bran d1 | 3.000-4.000 |
| Crude coco oil | 2.900-3.000 |
| Biofos TG21 | 1.800-1.820 |
| Limestone, fine | 1.500-1.300 |
| Salt | 0.500-1.310 |
| Lysine | 0.200-0.500 |
| Choline chloride, 25% | 0.200-0.301 |
| Vitamin premix swine | 0.100-0.200 |
| Mineral premix swine | 0.100-0.200 |
| Threonine | 0.060-0.065 |
| Anti-mold | 0.040-0.050 |
| Copper sulfate | 0.020-0.036 |
| DL-methionine | 0.020-0.025 |
| Anti-oxidant | 0.012-0.013 |
| Total | 100.00 |

TABLE 14
Nutrient content of the pre-starter diet for weanling swine

| Nutrients | % of Feed Content |
|---|---|
| Crude protein | 18.000-19.00 |
| Crude fat | 5.500-5.790 |
| Crude fiber | 2.000-2.150 |
| Calcium | 0.850-0.860 |
| Total phosphorus | 0.730-0.750 |
| Available phosphorus | 0.500 |
| ME, kcal/kg | 3200 |
| Lysine | 1.200-1.260 |
| Methionine | 0.280-0.300 |
| Methionine + cystine | 0.660-0.690 |
| Tryptohan | 0.220-0.250 |
| Threonine | 0.820-0.860 |

According to another embodiment of the invention, a cysteamine compound (such as 90 g of cysteamine HCl per tonnage of feed) is added to a basal feed diet that is given to growing pigs or pigs that are finishing their growth.

The basal feed diet fed to growing swine (also referred to herein as grower diet) preferably are formulated with a cysteamine compound of the invention and contain an energy:protein ratio of about 190. More preferably, the grower diet contains a 16.3% amount of crude protein.

In contrast, the basal feed diet fed to pigs that are finishing their growth (also referred to herein as finisher diet) are formulated with an energy:proteint ratio of about 215. More preferably, the finisher diet contains a 14% amount of crude protein.

An example of those materials that make up a basal feed diet for growing/finishing pigs is provided in Table 15 below. The nutrient content that makes up a basal feed diet for growing/finishing pigs is provided in Table 16 below.

TABLE 15
Composition of diet for growing/finishing swine

| | Hog Grower | Hog Finisher |
|---|---|---|
| Ingredients | % of Feed Content | % of Feed Content |
| Corn, yellow | 55.720-57.700 | 55.130-57.170 |
| Soybean meal, 46% | 16.480-21.100 | 12.000-13.600 |
| Copra meal | 10.000 | 10.200-12.000 |
| Rice bran d1 | 5.000 | 8.180-9.560 |
| Molasses | 2.000-4.230 | 5.000 |
| Crude coco oil | 1.790-2.000 | 2.000 |
| Biofos TG21 | 1.650-1.790 | 1.480-1.490 |
| Limestone, fine | 1.260-1.300 | 1.140-1.180 |
| Salt | 0.500 | 0.500 |
| Lysine | 0.390-0.430 | 0.340-0.390 |
| Choline chloride, 25% | 0.200 | 0.150 |
| Vitamin premix swine | 0.100 | 0.100 |
| Mineral premix swine | 0.100 | 0.100 |
| Threonine | 0.820-0.090 | 0.045-0.055 |
| Anti-mold | 0.050 | 0.050 |
| Copper sulfate | 0.040-0.050 | 0.025 |
| DL-methionine | 0.025 | 0.010-0.025 |
| Anti-oxidant | 0.010-0.013 | 0.012-0.120 |
| Total | 100.00 | 100.00 |

TABLE 16
Nutrient Content of diet for growing/finishing swine

| | Hog Grower | Hog Finisher |
|---|---|---|
| Nutrients | % of Feed Content | % of Feed Content |
| Crude protein | 15.500-17.100 | 13.300-14.700 |
| Crude fat | 4.900-5.360 | 5.840-6.160 |
| Crude fiber | 3.100-3.220 | 3.640-3.730 |
| Calcium | 0.850 | 0.750 |
| Total P | 0.750-0.770 | 0.740-0.760 |
| Available P | 0.500 | 0.450 |
| ME, kcal/kg | 3100 | 3100 |
| Lysine | 1.040-1.140 | 0.830-0.910 |
| Methionine | 0.250-0.285 | 0.190-0.220 |
| Methionine + cystine | 0.570-0.630 | 0.470-0.510 |
| Tryptohan | 0.180-0.210 | 0.140-0.170 |
| Threonine | 0.700-0.780 | 0.570-0.630 |

All patents, patent applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A feed formulation comprising a cysteamine compound and a crude protein level of at least about 14% of the formulation.

2. The feed formulation of claim 1, wherein the level of crude protein is about 14%.

3. The feed formulation of claim 1, wherein the level of crude protein is about 16%.

4. The feed formulation of claim 1, wherein the level of crude protein is about 18%.

5. The feed formulation of claim 1, wherein the cysteamine compound is cysteamine hydrochloride.

6. The feed formulation of claim 1, wherein the cysteamine compound consists of 50 to 1,000 g of the cysteamine hydrochloride per ton of the formulation.

7. The feed formulation of claim 1, further comprising a compound selected from the group consisting of: microbicides; antimicrobials; and vaccines.

8. The feed formulation of claim 1, further comprising anyone or combination of compounds selected from the group consisting of: yellow corn; soybean meal; copra meal; rice bran; molasses; crude coco oil; biofos TGZ1; limestone; salt; lysine; choline chloride; vitamins; minerals; threonine; anti-mold compounds; copper sulfate; DL-methionine; and anti-oxidants.

9. The feed formulation of claim 1, wherein the feed formulation is one suitable for livestock selected from the group consisting of: cattle, swine, sheep, goats, rabbits, mink, deer, alligator, snakes, and poultry.

10. A method for improving livestock productivity, wherein said method comprises administering to livestock an effective amount of a cysteamine compound and livestock feed.

11. The method of claim 10, wherein the livestock is selected from the group consisting of: cattle, swine, poultry, sheep, goats, and rabbits.

12. The method of claim 11, wherein the effective amount of cysteamine compound consists of 50 to 1,000 g of the cysteamine hydrochloride per ton of livestock feed.

13. The method of claim 10, further comprising the step of concurrently administering the cysteamine compound with other known compounds used to improve livestock health.

14. The method of claim 13, wherein the other known compounds are selected from the group consisting of: microbicides, antimicrobials, and vaccines.

15. The method of claim 10, wherein the administration of the cysteamine compound and livestock feed causes any one or combination of effects selected from the group consisting of: accelerated growth; enhanced fertility; enhanced appearance; and decreased volume of animal waste products.

16. A feed formulation for improving livestock productivity comprising an effective amount of a cysteamine compound.

17. The feed formulation of claim 16, wherein the cysteamine compound is cysteamine hydrochloride.

18. The feed formulation of claim 16, wherein the effective amount of the cysteamine compounds is 50-1,000 g per ton of feed formulation.

19. The feed formulation of claim 16, further comprising anyone or combination of compounds selected from the group consisting of: yellow corn; soybean meal; copra meal; rice bran; molasses; crude coco oil; biofos TGZ1; limestone; salt; lysine; choline chloride; vitamins; minerals; threonine; anti-mold compounds; copper sulfate; DL-methionine; and anti-oxidants.

20. The feed formulation of claim 16, wherein the feed formulation is one suitable for livestock selected from the group consisting of: cattle, swine, sheep, goats, rabbits, mink, deer, alligator, snakes, and poultry.
